(19) United States
(12) Patent Application Publication
(10) Pub. No.: US 2009/0078270 A1
(43) Pub. Date: Mar. 26, 2009

(54) Removable implant and implantation tool for male contraception

(75) Inventors: Peter A. Meier, Hamburg (DE); Axel Hentrich, Hamburg (DE)

Correspondence Address: BODNER & O'ROURKE, LLP, 425 BROADHOLLOW ROAD, SUITE 108, MELVILLE, NY, 11747, US

(73) Assignee: Ethicon, Inc., Somerville, NJ (US)

(21) Appl. No.: 11/904,266

(22) Filed: Sep. 26, 2007

(51) Int. Cl. A61F 6/02; A61B 17/58

(52) U.S. Cl. 128/843; 606/108

(57) ABSTRACT

A male contraception implant for insertion into the vas deferens of a patient includes a tubular member having an internal bore for receiving a guide wire, a bulbous head portion for anchoring the implant and to facilitate its removal in a contraception reversal procedure, a flexible neck portion attached to the enlarged head portion, and a main body portion having three widened segments for occluding the vas deferens. An implantation tool includes an elastic C-shaped slotted ring for holding an externalized portion of the vas deferens during the delivery of the implant.

### BACKGROUND OF THE INVENTION

1. Field of the Invention

This invention relates to implantable occlusion devices and more specifically relates to implantable male contraception devices and instruments used for the delivery of such contraception devices.

2. Description of the Prior Art

Definitive male contraception is most commonly achieved by a surgical vasectomy. While the vasectomy is an effective contraception method, the procedure is very often painful and has many long term effects that are not always desirable, but are accepted as other methods of definitive contraception are not readily available.

Conventionally, a vasectomy may be performed surgically by the traditional method of removing the vas deferens completely or by cutting and sealing the vas deferens, most commonly referred to as a “keyhole” or “no-scalpel” vasectomy. Patients undergoing either type of vasectomy should consider the-procedure permanent and not reversible. In some rare cases a vasectomy may be reversed by another surgical procedure known as a vasovasostomy; however, the success rate of completely returning the reproductive capabilities of the male with this procedure is poor at best. Post-vasectomy patients are commonly limited in activities for up to four weeks and are often prone to severe and chronic pain lasting years. The vasovasostomy has been an effective means of treating pain arising from vasectomy complications, but as with most microsurgical procedures, is extremely costly. Many males desiring an effective contraception method often hesitate when considering a vasectomy due to the essentially irreversible effects of the surgery and the potential for long lasting pain.

A new method of definitive male contraception utilizing an intra vas implant has been proven to be as effective as vasectomy procedures while having significantly less complications and long term side effects. The intra vas implant (IVI) operates in a manner similar to that of an intra uterine device (IUD) in females. To achieve complete contraception capabilities in males, the IVI is surgically implanted into the vas deferens, blocking the path of sperm from the testicles to the urethra. The conventional method of surgical IVI implantation in most cases is cumbersome and time consuming.

Two common implantable devices include an injectable implant and a sutured implant.

The injectable implant utilizes an injectable polymer. The polymer in its liquid form, commonly a microcellular polyurethane or medical grade silicone rubber, is injected into the vas deferens where it hardens within twenty (20) minutes. The resulting, hard spherical plug provides a barrier to sperm. This technique was developed in China and some 300,000 men have reportedly undergone the procedure. The injectable polymer contraception method may be reversible. However, to reverse such a method a surgical removal of the plug is necessary.

The sutured implant utilizes silicone plug pairs, commonly referred to as a “Shug”. A shug is implanted into each vas deferens and anchored to the wall by small sutures. Similar to the latter method, the plug acts as a barrier to sperm. The plug is theoretically reversible although it is reportedly still being developed.

The present invention discloses a novel implantable male contraception device that may be easily introduced and removed from a patient.

### OBJECTS AND SUMMARY OF THE INVENTION

It is an object of the present invention to provide an implantable device for male contraception and an implantation tool to aid in the delivery of the contraception device.

It is another object of the present invention to provide a male contraception implant that is removable.

It is yet another object of the present invention to provide a male contraception implant that may be easily inserted into the vas deferens of a patient.

It is a further object of the present invention to provide a male contraception implant that once implanted is flexible and comfortable.

It is yet a further object of the present invention to provide a male contraception implant which overcomes the inherent disadvantages of known male contraception implants.

In accordance with one form of the invention, a male contraception implant used to occlude the vas deferens of a male patient and prevent sperm travel is disclosed. The implant preferably includes an elongated tubular member having a central bore extending axially therethrough. The tubular member has basically three sections: an enlarged head portion which protrudes out of the incision in the vas deferens to aid in the removal of the implant in a reverse contraception procedure, a relatively thin tubular neck portion affixed to the head portion, and a main body portion formed of at least one widened segment, but more preferably with three widened segments, which is received by and occludes the vas deferens.

In accordance with another form of the present invention, an implantation tool to aid in the delivery of a contraception device includes a slotted ring, preferably oval or circular in shape. During the implantation procedure, an incision is made in the patient's scrotum, and the vas deferens is exposed therethrough and placed about the slot in the ring. The exposed vas deferens, held in place by the ring, is more easily accessible to the physician. A small incision is now made across the longitudinal line of the vas deferens, and a guide wire is inserted into the vas deferens. The implant, slideably mounted on the guide wire, is now placed in the vas deferens in a desired position, with the enlarged head portion of the implant exposed. The incision in the vas deferens about the protruding head of the implant is then sealed. Then, the exposed vas deferens is removed from the implantation tool and reinserted through the incision in the scrotum. The scrotum incision is then sealed.

A preferred form of the male contraception implant and implantation tool, as well as other embodiments, objects, features and advantages of this invention, will be apparent from the following detailed description of illustrative embodiments thereof, which is to be read in conjunction with the accompanying drawings.

### BRIEF DESCRIPTION OF THE DRAWINGS

FIG. 1 is a perspective view of the male contraception implant formed in accordance with a first embodiment of the present invention.

FIG. 2A is a side view of the male contraception implanted formed in accordance with a second embodiment of the present invention.

FIG. 2B is a top view of the male contraception implant formed in accordance with a second embodiment of the present invention.

FIG. 3 is semi-opaque view of the male contraception implant formed in accordance with the present invention being inserted into the vas deferens, as well as a first embodiment of an implantation tool formed in accordance with the present invention.

FIG. 4 is semi-opaque view of the male contraception implant formed in accordance with the present invention within the vas deferens.

FIG. 5 is a bottom isometric view of a second embodiment of an implantation tool formed in accordance with the present invention.

FIG. 6 is a top isometric view of the second embodiment of the implantation tool shown in FIG. 5.

### DETAILED DESCRIPTION OF THE PREFERRED EMBODIMENTS

The present invention is an implantable male contraception device 2 that may easily be introduced and, if necessary, removed from a patient, and an implantation tool 4 that aids in the delivery of the implant 2.

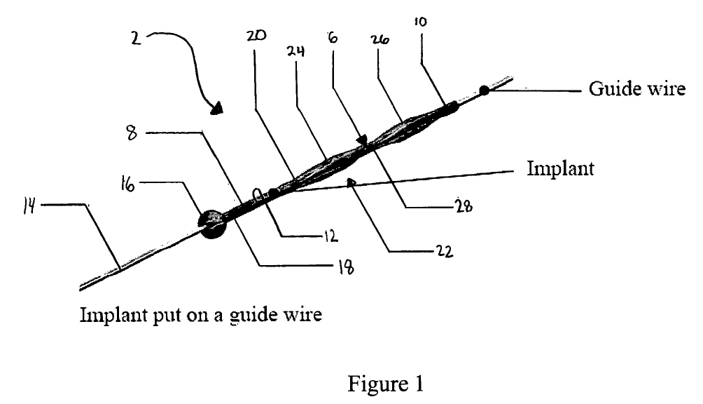

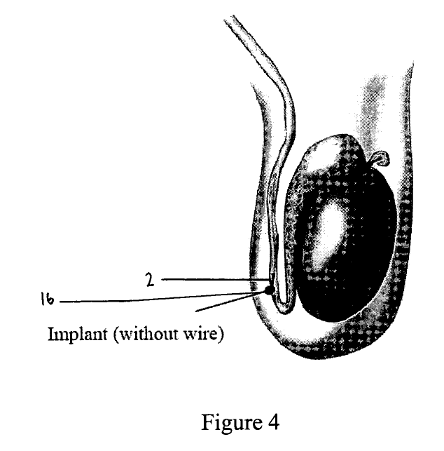

As shown in FIGS. 1, 2A, 2B, 3 and 4 of the drawings, it will be seen that a preferred form of the contraception implant 2 of the present invention used to occlude the vas deferens of a male patient and prevent sperm travel therein includes an elongated tubular member 6 having a first axial end 8 and a second axial end 10 situated opposite the first axial end 8, and an internal bore 12 extending axially therethrough along the longitudinal axis from the first axial end 8 to the second axial end 10. The bore 12 is provided for receiving a guide wire 14 therein so that the implant 2 is slideable on the guide wire 14 and delivered thereby to the vas deferens of the patient during the implantation procedure.

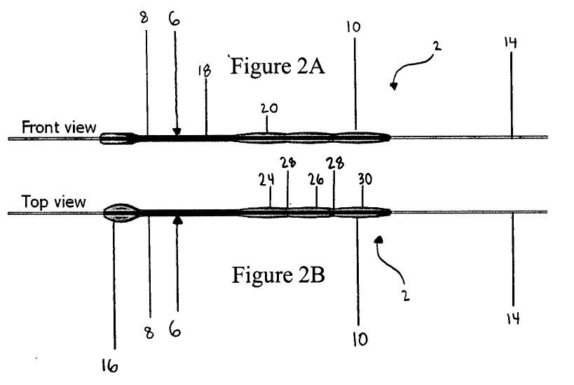

The tubular member 6 of the implant 2 basically includes three sections along its axial length, each section being defined by a particular shape, or width measured radially from the longitudinal axis of the tubular member. More specifically, the tubular member includes a head portion 16 situated at or near the first axial end 8 of the tubular member 6. In the first embodiment of the present invention shown in FIG. 1, this head 16 portion of the implant 2 is formed as a spherical bead or bulbous member. The head portion 16 preferably has a diameter which is greater than the diameter or radial widths of the other two sections of the tubular member 6. In the second embodiment of the invention shown in FIGS. 2A and 2B, the head portion 16 is flattened, rather than spherical, and has generally convex, outwardly curved, opposite lateral sidewalls in mirrored symmetry to each other, and generally parallel opposite top and bottom walls joined perpendicularly to the lateral sidewalls.

The tubular member 6 of the implant 2 further includes a neck portion 18 which is situated adjacent the head portion 16 along the longitudinal axis of the tubular member 6. The neck portion 18 is a flexible, relatively thin cylindrical section of the tubular member 6, having an outer diameter which is preferably less than the diameter or width of the head portion 16 and the overall width or diameter of the third section, the main body portion of the implant 2, as will be described.

The main body portion 20 of the tubular member 6 of the implant 2 is the section which is designed to occlude the vas deferens. This section is received interiorly along a portion of the vas deferens to prevent the passage of sperm therethrough. The main body portion 20 is attached to the other axial end of the neck portion 18 along the longitudinal axis of the tubular member 6. The main body portion 20 includes at least one widened segment 22, but more preferably includes a plurality of widened segments, measured from the longitudinal axis of the elongated tubular member 6 of the implant 2.

More specifically, and as can be seen from FIG. 1 of the drawings which shows a first embodiment of the implant 2 of the present invention, the main body portion 20 of the implant 2 includes first and second wide body segments 24,26 interconnected by a narrower width portion 28. In FIGS. 2A and 2B, a second embodiment of the implant 2 of the present invention is illustrated as having a main body section 20 with first, second and third wide body segments 24,26,30, adjacent segments being interconnected by a narrower width portion 28.

It can also be seen from a comparison of FIGS. 1 and 2A that the neck portion 18 in the second embodiment (FIG. 2A) is longer than the neck portion 18 of the first embodiment (FIG. 1).

The second embodiment of the implant 2 shown in FIGS. 2A and 2B, with its longer neck 18 and flattened head 16 and three segmented main body 20, is envisioned to provide more flexibility and more safety than the first embodiment of the implant 2 shown in FIG. 1, although each of the first and second embodiments will perform the desired function of occluding the vas deferens and preventing sperm flow therein.

With either embodiment, the first most distal widened portion segment 30 occludes the duct (i.e., vas deferens, or more generally, any anatomical vessel). However, the second widened body segment 26 will block any sperm that leak past the first segment 30. The third widened portion segment 24 of the second embodiment which is closest to the neck portion 18 provides even greater safety.

The preferred overall diameter of the implant 2 is less than about 1.2 millimeters. This size allows for even small incisions to be made in the vas deferens than conventionally, and the incision is expected to heal completely and rapidly. The enlarged head 16 of the implant 2 remains outside the vas deferens and provides a means to find the implant 2 in the event the patient wishes to reverse the contraception. The enlarged head 16, outside the vas deferens, also helps prevent migration of the implant 2 within the duct, without requiring sutures. The implant 2 is preferably constructed of medical grade silicone and may be formed by injection molding or other similar method. The use of silicone yields a flexible, comfortable implant 2 that does not induce the sensation to the patient of the presence of a foreign body.

The implant 2 may be inserted over a guide wire 14 through a small incision into the vas deferens of a patient. The main body portion 20 near the second axial end 10 of the implant 2 is inserted first, followed by the neck portion 18 near the first axial end 8. Proper placement of the implant 2 calls for the head 16 to remain outside the vas deferens. The widened segments 22 will form an interference fit with the walls of the vas deferens, thus securing the implant 2 within the vas deferens and forming a seal with the walls to prevent sperm flow past the device. The guide wire 14 may then be removed and the small incision will heal naturally. To remove the implant 2, the head 16 may be grasped by hand or instrument, and withdrawn from the vas deferens without any significant surgical intervention such as required by the conventional contraception implants and procedures.

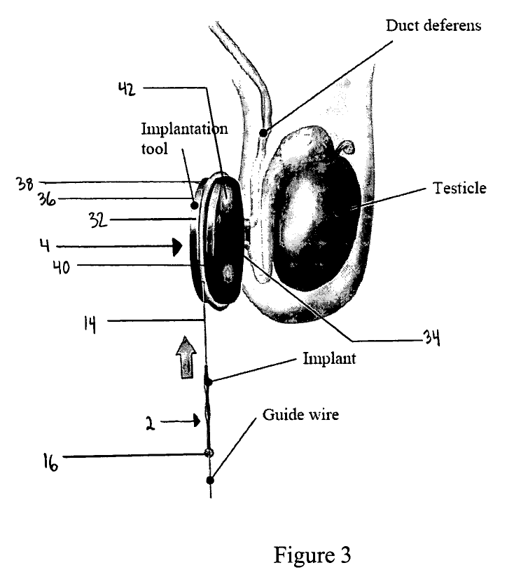

FIGS. 3, 5 and 6 illustrate two embodiments of an implantation tool 4 constructed in accordance with the present invention which aid in the delivery of the contraception implant 2. Because the vas deferens is slippery, it is difficult to grasp during implant 2 delivery. The implantation tool 4 of the present invention described herein maintains the duct in a secure position for the physician so that proper insertion of the contraception device may be achieved quickly and easily.

The first embodiment of the implantation tool 4 is shown in FIG. 3. The tool includes a support 32 for the vas deferens in the form of a circular, oval, elliptical or other curved body. More specifically, the body 32 includes opposite front and rear faces 34,36, and a curved sidewall 38 extending circumferentially about the support 32 between the front and rear faces 34,36. An open slot 40 or recess is formed in the surface of the curved sidewall 38, the dimensions (depth and width) of which are selected to allow an externalized (exposed) portion of the vas deferens to be at least partially received therein.

Preferably, the front face or rear face, or both faces, may include either one or more protrusions 42 extending outwardly therefrom, or one or more depressions, or again both protrusions and depressions provided on either or both faces. The protrusions 42 and depressions help the physician grasp the implantation tool 4 as he or she is placing the externalized vas deferens on the support 32, or inserting the guide wire 14 or positioning the implant 2 in the patient in the exposed portion of the vas deferens supported thereby.

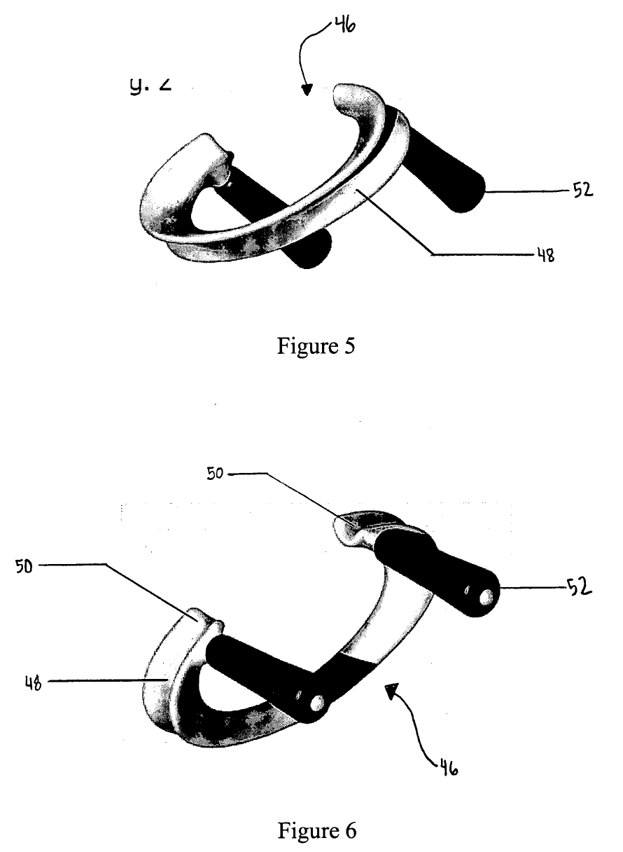

FIGS. 5 and 6 show the second embodiment of the implantation tool 4 of the present invention. In this embodiment, the support is in the form of an elastic, slotted ring 44. More specifically, the support includes a C-shaped body 46 made from a resilient material. On the outer surface thereof is formed a cavity or slot 48, again like the first embodiment, dimensioned to at least partially receive therein a portion of the externalized vas deferens. The open ends 50 of the ring preferably include handles 52 protruding perpendicularly therefrom that are graspable by the physician so that the two ends of the implantation tool 4 may be elastically brought together to decrease the effective diameter of the device while placing the vas deferens around its circumference.

The implantation procedure, using either embodiment of the implantation tool 4 of the present invention, will now be described. An incision is made in the patient's scrotum to expose the vas deferens. A section of the vas deferens is externalized (i.e., a portion of the vas deferens is pulled out of the scrotum through the incision) and placed around the implantation tool 4 to hold the otherwise slippery vas deferens in position. With the embodiment shown in FIGS. 5 and 6, the two ends 50 of the insertion tool 4 are elastically brought together to decrease the effective diameter of the device while placing the vas deferens around its circumference.

After a small incision is made across the longitudinal line of the vas deferens, a guide wire 14 is inserted into the vas deferens. With the help of the guide wire 14, the implant 2 device, either slideably pre-mounted on the guide wire 14 or placed on the guide wire 14 at this time, is maneuvered on the guide wire 14 into the vas deferens, with the enlarged head 16 remaining outside the semen duct (i.e., the vas deferens). After the implant 2 is properly delivered, the guide wire 14 is withdrawn. The incision in the vas deferens should heal naturally about the neck portion 18 of the implant 2 or may be sealed by the physician, leaving the enlarged head portion 16 exposed. The vas deferens is removed from the support and replaced in the scrotum. The incision in the scrotum is then sealed.

An alternative procedure would be to make a second incision in the externalized vas deferens, which second incision is spaced apart longitudinally from the first incision. The guide wire 14, with the implant 2 slidably attached thereto, is inserted into the vas deferens through the first incision, as described previously. However, instead of removing the guide wire 14 back out of the first incision after the implant 2 is properly positioned within the vas deferens, in this alterative procedure, the insertion end of the guide wire 14 is pulled through the vas deferens and out of the second incision. The implant 2, frictionally attached to the guide wire 14, is pulled into the vas deferens through the first incision. However, the enlarged head portion 16 of the implant 2 engages the outer surface of the vas deferens at the first incision and prevents the implant from following the guide wire 14 completely through the vas lumen. The engagement of the enlarged head 16 with the outer surface of the vas deferens overcomes the frictional force retaining the implant 2 to the guide wire 14 and thereby causes the implant 2 to be released from the guide wire 14 in the proper position in the vas lumen. The guide wire 14 is completely pulled through the second incision formed in the vas deferens, leaving the implant 2 behind and positioned in the vas lumen.

Since the implant 2 is preferably less than 1.2 millimeters in overall diameter, only a minor incision needs to be made in the vas deferens. Furthermore, because of the flexibility and shape of the implant 2, there is less irritation of the tissue.

The contraception device of the present invention may be implanted in an easy and gentle way. Preferably, as mentioned previously, the implant 2 is formed from a medical grade silicone. The implant 2 may be easily manufactured by injection molding.

The implantation tool 4 of the present invention preferably has a dimension of about 10 millimeters to about 20 millimeters. The tool 4 acts as a holder for the semen duct (vas deferens) and assists the physician by holding the vas deferens in position during the delivery of the implant 2.

It should be realized that the implant device 2 and implantation tool 4 of the present invention described herein are not limited to use in occluding the vas deferens. It is foreseen that the implant 2 and implantation tool 4 may be useful as not only a means for male contraception, but also may be used for occluding blood vessels and other body lumens with implants having one end protruding through the vessel wall. Other areas of application include use of the implant 2 and implantation tool 4 in the treatment of aneurisms or for the occlusion of fallopian tubes, or in the treatment of myomas.

The present invention overcomes the disadvantages inherent with the conventional designs by decreasing the complexity of implantation and the removal procedure. Utilization of the present invention provides a reversible, comfortable and definitive contraception method that can be performed by any urologist.

What is claimed is:

1. An implant for at least partial insertion in a vessel of a patient and for occluding the vessel, which comprises:
an elongated tubular member, the tubular member having a first axial end and a second axial end situated opposite the first axial end, and an internal bore extending axially therethrough at least partially along the axial length thereof, the tubular member including an enlarged head portion for residing outside the vessel when the implant is at least partially inserted into the vessel, a flexible neck portion attached to the enlarged head portion, and a main body portion attached to the neck portion, the main body portion having at least one widened body segment for occluding the vessel, the main body portion being positionable within the vessel.

2. An implant as defined by claim 1, wherein the main body portion has a plurality of widened body segments including adjacent widened body segments, the adjacent widened body segments being operatively coupled to each other.

3. An implant as defined by claim 2, wherein the main body portion includes at least one narrowed body portion, the at least one narrowed body portion being interposed between the adjacent widened body segments and being interconnected to the adjacent widened body segments to operatively couple the adjacent widened body segments together.

4. An implant as defined by claim 1, wherein the neck portion includes a relatively thin, flexible portion.

5. An implant as defined by claim 1, wherein the enlarged head portion includes a generally bulbous member.

6. An implant as defined by claim 1, wherein the enlarged head portion includes generally convex, outwardly curved, opposite lateral sidewalls, and generally parallel opposite top and bottom walls joined to the outwardly curved, lateral sidewalls.

7. A male contraception implant for being at least partially inserted into the vas deferens of a patient and for occluding the flow of semen therethrough, which comprises:
an elongated tubular member, the tubular member having a first axial end and a second axial end situated opposite the first axial end, and an internal bore extending axially therethrough at least partially along the axial length thereof, the tubular member including an enlarged head portion for residing outside the vas deferens when the implant is at least partially inserted into the vas deferens, a flexible neck portion attached to the enlarged head portion, and a main body portion attached to the neck portion, the main body portion having at least one widened body segment for occluding the vas deferens, the main body portion being positionable within the vas deferens.

8. An implantation tool for holding an externalized duct of a patient during an implantation procedure, which comprises:
a support member having a curved outer surface, the curved outer surface having a groove formed therein for at least partially receiving the externalized duct of the patient during the implantation procedure.

9. An implantation tool as defined by claim 8, wherein the support member includes a front face and a rear face opposite the front face, and a sidewall interposed between the front face and the rear face, the sidewall having the curved outer surface and the groove formed therein.

10. An implantation tool as defined by claim 9, wherein at least one of the front face and the rear face of the support member includes at least one of a protrusion extending outwardly therefrom and a depression formed therein to facilitate grasping the support by a physician.

11. An implantation tool as defined by claim 8, wherein the support member includes a C-shaped elastic ring having at least a partial circumference, the ring having opposite first and second ends spaced apart from each other by a spacing, the ring being bendable to adjust the spacing between the first and second ends in order to change the at least partial circumference of the C-shaped ring, the curved outer surface and groove formed therein extending at least partially between the first end and the second end of the ring.

12. An implantation tool as defined by claim 11, wherein the support member further includes first and second handles, the first handle extending outwardly from the first end of the C-shaped ring, and the second handle extending outwardly from the second end of the C-shaped ring, the first and second handles being provided for grasping by a physician during the implantation procedure.

13. A method of occluding a vessel using an implant as defined by claim 1, which comprises the steps of:
introducing a guide wire into the vessel of the patient;
advancing the implant over the guide wire at least partially into the vessel; and
removing the guide wire from the vessel.

14. A method of contraception using a male contraception implant and an implantation tool, which comprises the steps of:
forming an incision in the scrotum of a patient;
pulling through the incision in the scrotum a portion of the patient's vas deferens to provide an externalized portion of the vas deferens;
looping about the implantation tool the externalized portion of the vas deferens, the implantation tool including a support member having a curved outer surface, the curved outer surface having a groove formed therein for at least partially receiving the externalized portion of the vas deferens;
forming an incision in the externalized portion of the vas deferens looped about the implantation tool;
at least partially inserting the implant in the externalized portion of the vas deferens looped about the implantation tool through the incision formed in the vas deferens;
removing the externalized portion of the vas deferens from the implantation tool; and
repositioning the externalized portion of the vas deferens in the patient's scrotum through the incision made therein.

15. A method as defined by claim 14, wherein the implantation tool further includes a C-shaped elastic ring having at least a partial circumference, the ring having opposite first and second ends spaced apart from each other by a spacing, the ring being bendable to adjust the spacing between the first and second ends in order to change the at least partial circumference of the C-shaped ring, the curved outer surface and groove formed therein extending at least partially between the first end and the second end of the ring; and wherein prior to the step of looping about the implantation tool the externalized portion of the vas deferens, the method further comprises the step of adjusting the spacing between the first and second ends of the C-shaped ring to change the at least partial circumference of the C-shaped ring.

16. A method as defined by claim 14, wherein the implant includes an elongated tubular member, the tubular member having a first axial end and a second axial end situated opposite the first axial end, and an internal bore extending axially therethrough at least partially along the axial length thereof, the tubular member including an enlarged head portion for residing outside the vas deferens when the implant is at least partially inserted into the vas deferens, a flexible neck portion attached the enlarged head portion, and a main body portion attached to the neck portion, the main body portion having at least one widened body segment for occluding the vas deferens, the main body portion being positionable within the vas deferens; wherein prior to the step of at least partially inserting the implant in the externalized portion of the vas deferens looped about the implantation tool, the method further comprises the step of at least partially inserting a guide wire in the externalized portion of the vas deferens looped about the implantation tool through the incision formed in the vas deferens; wherein the step of at least partially inserting the implant in the externalized portion-of the-vas-deferens looped about the implantation tool further comprises the step of slideably advancing the implant on the guide wire so that at least the main body portion thereof is inserted in the externalized portion of the vas deferens looped about the implantation tool through the incision formed in the vas deferens; and wherein prior to the step of removing the externalized portion of the vas deferens from the implantation tool, the method further comprises the step of removing the at least portion of the guide wire inserted into the vas deferens looped about the implantation tool, leaving the implant at least partially inserted in the externalized portion of the vas deferens.
